(19) United States
(12) Patent Application Publication
(10) Pub. No.: US 2008/0033484 A1
(43) Pub. Date: Feb. 7, 2008

(54) Method and device for relieving pain

(76) Inventor: Jiman He, Pawtucket, RI (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 11/497,923

(22) Filed: Aug. 1, 2006

(51) Int. Cl. A61B 17/00

(52) U.S. Cl. 606/204

(57) ABSTRACT

Methods of providing relief from pain in a patient include applying direct pressure on at least one of the sciatic nerves of the patient. Devices for performing the methods are further disclosed.

### BACKGROUND OF THE INVENTION

1. Field of the Invention

This invention relates generally to methods for relieving pain, and more particularly to a method for relieving pain by applying pressure to a patient's sciatic nerve.

2. Discussion of Related Art

At any given time, people are subject to the experience of pain. In most instances, pain cannot be rapidly relieved. Overcoming pain in a fast and convenient way, therefore, is a worthwhile goal that has yet to be achieved despite considerable research. The presently available analgesics do not adequately or quickly relieve patients experiencing pain. Many commonly used medicines, both over-the-counter and prescription drugs, take considerable time to relieve pain and may have well-known serious side effects. There are alternative pain relief applications not involving drug analgesics, such as acupuncture, transcutaneous electrical nerve stimulation (“TENS”), and interferential stimulation (“IFS”), to name a few. However, these alternative applications may take a considerable amount of time to take effect, are costly, may require special settings or require the skills of a trained practitioner to apply them, and may be applicable in limited circumstances and provide limited pain relief.

### SUMMARY OF THE INVENTION

One aspect of the invention is directed to a method of providing relief from pain in a patient. In a certain embodiment, the method comprises applying direct pressure on at least one of the sciatic nerves of the patient. Other embodiments of the method may further comprise applying pressure for a period of time, wherein pressure is applied for a period of time extending between twenty seconds and ten minutes. The relief from pain experienced by the patient may last at least thirty minutes. The method may be achieved by applying direct pressure on at least one of the sciatic nerves of the patient by using a person's hand. In other embodiments, the method may be achieved by applying direct pressure on at least one of the sciatic nerves of the patient by utilizing a device having a small surface configured to engage the sciatic nerve. The method is particularly suited for relieving pain caused by a medical condition. In a certain embodiment, the medical condition may be selected from a group consisting of a renal condition, a dental condition and a tumor condition.

Another aspect of the invention is directed to a method of providing relief from pain in a patient caused by a medical condition. In one embodiment, the method comprises: applying a first pressure relieving device on at least one of the sciatic nerves of the patient; manipulating the first pressure relieving device to increase the pressure on the at least one of the sciatic nerve of the patient; and applying the first pressure relieving device at an increased pressure for a period of time. Embodiments of the method may further include applying a second pressure relieving device on the other of the sciatic nerves of the patient, manipulating the second pressure relieving device to increase the pressure on the other of the sciatic nerves of the patient, and applying the second pressure relieving device at an increased pressure for a period of time. The device may be configured with a small surface to apply pressure on the sciatic nerve. The medical condition may be selected from a group consisting of a renal condition, a dental condition and a tumor condition.

Yet another aspect of the invention is directed to a pressure application assembly comprising a belt configured to wrap around a leg of a patient, and a device coupled to the belt. The device is configured to apply pressure to a sciatic nerve of the patient. In certain embodiments, the device further comprises an apparatus, coupled to the belt, to increase the application of pressure of the device against the sciatic nerve of the patient. The apparatus comprises an inflatable bladder and a bulb in fluid communication with the inflatable bladder to deliver air under pressure to the inflatable bladder. The device may be configured to comprise a small surface configured in the shape of an arc.

A further aspect of the invention is directed to a pressure application device configured to apply pressure to a sciatic nerve of the patient. The pressure application device may be configured to comprise a small surface to engage the sciatic nerve of the patient. In a certain embodiment, the small surface of the device is configured in the shape of an arc.

### DESCRIPTION OF THE DRAWINGS

For a better understanding of the invention, reference is made to the drawing figures which are incorporated herein by reference and in which:

FIG. 1 is a perspective view of a pressure application device of an embodiment of the invention;

FIG. 2 is a view showing the pressure application device prior to its employment on a patient;

FIG. 3 is a perspective view of a pressure application device of another embodiment of the invention; and

FIGS. 4-7 are perspective views of pressure application devices of further embodiments.

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

It has been reported that the stimulation of peripheral nerves elevates a patient's pain threshold. Although the physiological mechanism is not clear, peripheral nerve stimulation has been used in clinical practice for analgesia, as in the use of the aforementioned TENS application, for example. One popular explanation is a “Gate Control” theory of pain, which proposes that stimulation of large-diameter afferent fibers might reduce pain. This theory proposes that stimulation of large-diameter afferent fibers inhibits second-order neurons in the dorsal hom, and prevents pain carried by small-diameter fibers from reaching higher brain centers. Another favored explanation involves the use of opiates to treat and deaden a patient's nerve system.

The sciatic nerve trunk is the largest and longest in the human body. The sciatic nerves extend from a person's lower back through each leg. Specifically, the sciatic nerve runs from the sacral plexus to the pelvis and upper leg. In humans, conditions associated with the sciatic nerve, such as pressure caused by a bulging disc in the lower back, is commonly viewed as a leading cause of pain in the lower back and legs.

In an embodiment of the invention, a method of relieving pain associated with certain medical conditions, such as renal-, dental- and tumor-related conditions is disclosed herein. Specifically, in one embodiment, pressure is directly applied simultaneously to the sciatic nerves at the back of the patient's legs while the patient lies in a prone position, for example.

The methods described herein may be applied in any position, including a standing position. Pressure may be applied by using parts of a person's hands, such as thumbs and/or fingers, fists, or by using a pressure application device, which device shall be described herein in greater detail below. It has been shown that the application of pressure on the patient's sciatic nerves for a period of time produces short term analgesia for pain located throughout the body. In one embodiment, the application of direct pressure on the sciatic nerve or nerves of the patient may range between twenty seconds and ten minutes. With some patients, quick relief from pain may occur at approximately twenty seconds after the application of direct pressure on a patient's sciatic nerve. However, pain relief is more likely to begin within two to ten minutes after applying direct pressure. More specifically, the application of direct pressure may be applied between two and three minutes. However, given the differences in a particular patient's pain threshold and physiology, the time period of the application of direct pressure on the patient's sciatic nerve may vary from patient to patient.

Specifically, in a confidential study conducted by the inventor involving two hundred thirty-five patients, fifty-eight patients had dental-related pain, thirty-four had renal-related pain, forty-one had tumor-related pain, and the remaining ninety-five patients having a variety of afflictions were from the emergency room. Of the two hundred thirty-five patients, two hundred twenty-four completed treatment. Of the two hundred twenty-four patients who completed treatment, one hundred nineteen received a placebo treatment and one hundred sixteen received treatment in accordance with the teachings of the methods of the invention. The study was a singly blinded, randomized and placebo-controlled trial. The patients were informed that the experiments were designed to test whether the methods are effective in human pain relief. The attending physician or assistant instructed the patients to evaluate pain using a visual analogue scale (“VAS”), with pain scaled from zero to ten wherein zero indicated no pain and ten indicated the most pain. Relief of pain was analyzed for short time periods of time, e.g., zero, five or ten minutes, with one overall group of patients being analyzed for longer time periods, e.g., zero, ten, twenty, thirty, forty, fifty or sixty minutes. Baseline VAS scores and age of patients were compared between the placebo groups and the actual test groups. A “placebo press” involved applying pressure simultaneously to both legs of the patient and at a parallel spot on the fronts of the patient's legs while the patient lied in a supine position. Categorical data were analyzed by using chi-squared tests, or Fisher Exact tests. Changes from the baseline for pain relief were assessed by using paired t-tests, both for the actual test groups and for the placebo groups. Comparisons to the placebo groups were performed using an analysis of covariance procedure, with adjustment for baseline VAS score, sex and age. All tests performed were two-sided, and a p-value of less than 0.05 was considered as statistically significant.

During the study, ninety-three patients enrolled in the application of pressure on the patients' sciatic nerves. For the test in renal disease patients, both the placebo group and the “sciatic press” group had twelve patients each. For the test in dental disease patients, each group (i.e., sciatic press and placebo) had twenty-one patients. For the test in tumor patients, the sciatic press group had fourteen patients and the placebo group had eleven patients. Chart 1 shows the test results for renal diseases:

Chart 2 shows the test results for dental diseases:

And finally, Chart 3 shows the test results for tumor-related diseases:

As shown, the test scores at zero, five and ten minutes were significantly lower after sciatic nerve pressure than at baseline with all three disease groups. For the placebo treatments, the VAS scores were also significantly lower than the actual treatments at baseline at some time points.

A method of providing relief from pain in a patient may be applied to relieve pain caused by other diseases as well. For example, ninety-five emergency room patients with pain caused by different diseases were tested. Specifically, there were forty-eight patients placed in the placebo group and 47 patients in the sciatic press group. Chart 4 shows the test results for emergency room patients:

As shown, the VAS pain score was significantly lower after the application of sciatic nerve pressure than at baseline. For the placebo treatments, the VAS scores were also lower than the actual treatments at baseline. However, pain relief with the emergency room patients is reduced by forty-three percent, which is lower than that seen in dental disease patients and in the tumor patients (about seventy percent for both). Nearly thirty percent of patients reported no pain relief in the experiment with emergency room patients. One possible reason for the high percentage of patients experiencing no relief is that many of the doctors and/or assistants in emergency rooms were new to the application of the method and were not applying the method correctly.

To test how long the duration of pain relief lasted in patients receiving the application of pressure on their sciatic nerves, forty-seven patients with pain from dental diseases, renal diseases and tumors were tested for a period of one hour. The placebo group had twenty-one participants and the sciatic press group had nineteen participants. Chart 5 illustrates the test results for the sixty minutes test:

As shown, the VAS pain scores were significantly lower at all time points after the sciatic nerve pressure application than at baseline. VAS scores were also lower than at baseline at all time points in a placebo treatment. After controlling for baseline pain, sex, age and diseases, mean VAS scores were statistically significant between the sciatic press application group and the placebo group at all time points. Pain relief ratings averaged more than seventy percent for dental and tumor patients, approximately fifty percent for renal patients, and approximately forty-three percent of the patients having various pathologies. On average, the analgesia lasted more than sixty minutes. The results show that pressure applied to the patient's sciatic nerves may achieve short term, but significant deadening or absence of pain in the patient.

The methods of relieving pain disclosed herein may be applied to patients having other types of discomfort, such as discomfort caused by cancer, belly swelling, high fever and chemotherapy, to name a few. To test for the effectiveness of the methods of relieving such discomfort, patients were randomly grouped into one of two groups, namely a direct press group and a placebo group. The application of the direct press method was applied for 100 seconds, with the method be performed by using fists on the sciatic nerves. Chart 6 illustrates the test results:

Referring now to the drawings, and more particularly to FIG. 1, there is generally indicated at 10 a device for applying discrete pressure on a patient's sciatic nerve in accordance with an embodiment of the invention. As mentioned above, the methods described herein may be achieved by using a person's hand (including the thumb, finger(s), or the fist) to apply pressure on the sciatic nerve. However, the device 10 shown and described herein may be employed to apply acute pressure on the sciatic nerve, thereby achieving better results.

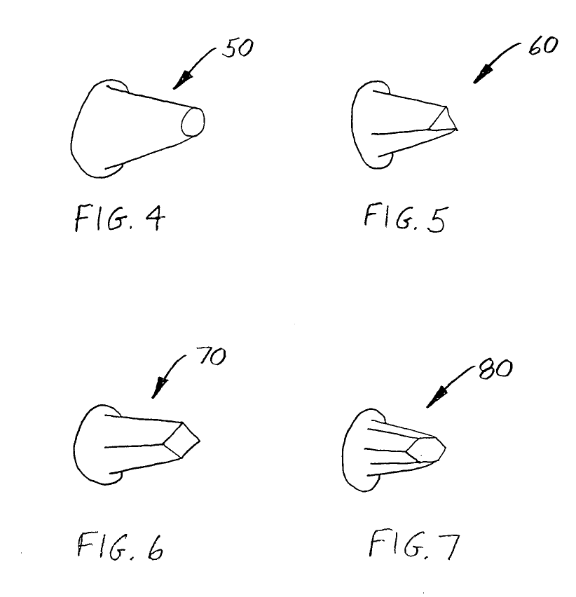

The pressure application device 10 is configured to be applied directly to the back of the patient's legs at the locations of the patient's sciatic nerves. In another embodiment, the device may be configured to be applied to a patient's peroneal nerves, which are located at the lower legs of the patient. As shown, the device 10 is generally trapezoidal in cross section, having a small inner diameter surface 12 and a larger outer diameter surface 14. The small surface 12 has a surface area sufficiently small so as to produce a large pressure when applying the device 10 to the patient's sciatic nerve. In one embodiment, the surface area of the small surface 12 is less than sixty-four cm². Although the small surface 12 is illustrated in FIG. 1 as being curved and relatively long in length, as will be discussed below and with reference to FIGS. 4-7, the small surface may take any shape that is sufficient to apply the direct pressure required by the methods described herein. Depending on the size of the patient's upper leg, the small surface 12 may be sized and configured to optimize the application of direct pressure.

The outer surface 14 is larger in surface area so as to provide a suitable surface to apply the device 10 by hand or with an apparatus designed to apply the device against the sciatic nerve of the patient.

As mentioned above, the surface area of the small surface 12 may be varied, as well as the radius of the curve constituting the small surface, to produce pressure on a small area of the leg. In one embodiment, the curve or radius of the small surface 12 may be chosen based on the size of the patient's upper leg. The pressure on the sciatic nerves (and the peroneal nerves) may be decreased when the surface area of the inner diameter surface is increased, which may result in poor pain relief. When less area is pressed against the sciatic nerve, a higher force is produced, which results in greater pain relief. In one embodiment, the device 10 may be fabricated from any material rigid enough to apply the pressure required to perform the methods discussed above. For example, a dense polymeric material may be employed.

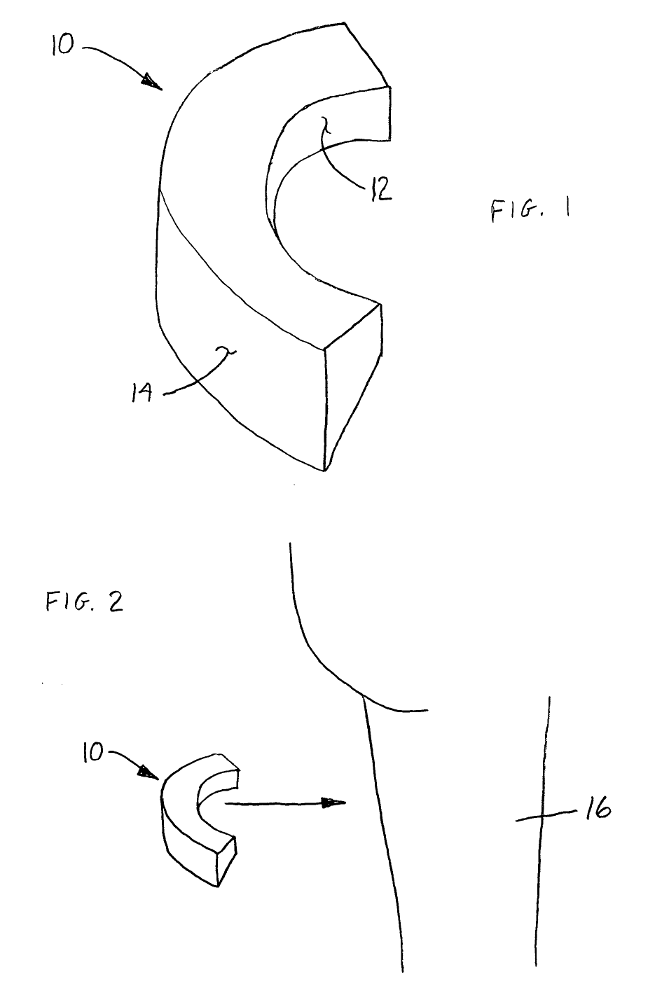

FIG. 2 illustrates the application of the device against a back of a patient's leg 16 in the area of the sciatic nerve. In a certain embodiment, pressure may be applied with the device 10 on the sciatic nerve of one of the patient's legs. However, as discussed above, better results are achieved when applying pressure on both of the sciatic nerves of the patient's legs. Also, better results, i.e., greater pain relief, are achieved when applying pressure on the sciatic nerves of the patient rather than on the peroneal nerves.

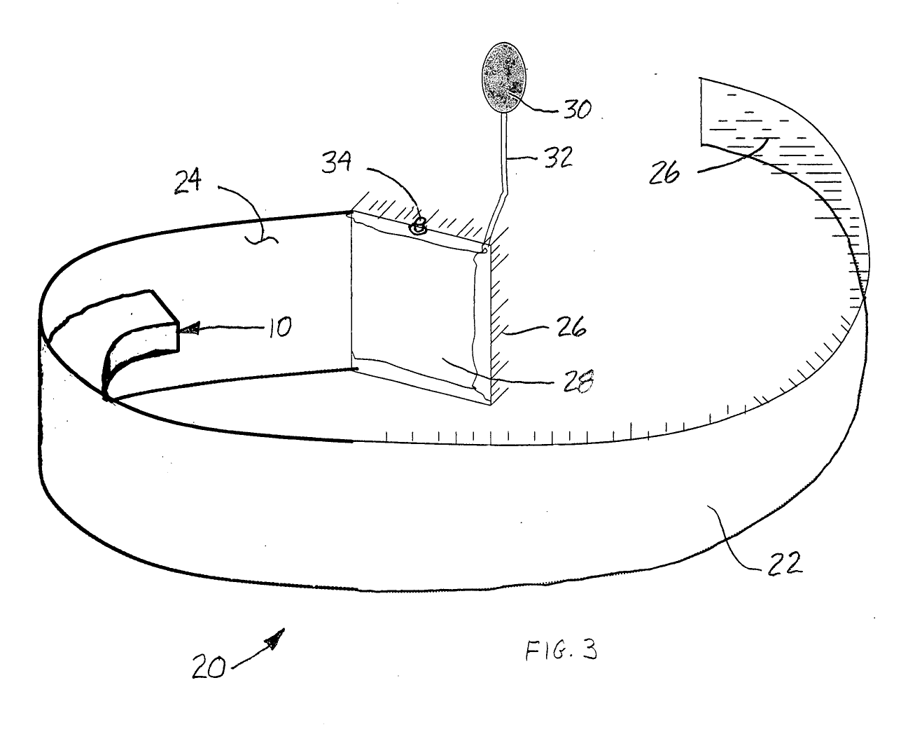

Turning now to FIG. 3, the device 10 is illustrated as part of a pressure application assembly, which is generally indicated at 20. As shown, the assembly 20 may include an elongate belt 22 configured to be secured around the patient's leg. Although a relatively flexible belt is illustrated in FIG. 3, it should be understood that a more rigid structure may be employed, e.g., a more rigid structure that is generally triangular-shaped, having ends that may be pulled to force the device 10 against the sciatic nerve of the patient. The device 10 may be suitably secured to the belt 22 so that the large diameter surface 14 is disposed adjacent an inner surface of the belt. For example, a strong adhesive or mechanical connection may be used to secure the device 10 to the belt 22. However, any suitable method may be employed to secure the device 10. The belt 22 may be configured with hook and loop fastener material 26 to tightly secure the belt around the patient's leg. In one embodiment, an inflatable bladder 28 may be provided to take up the length of the belt 22 thereby increasing the pressure applied by the device 10 on the patient's sciatic nerve. A bulb 30 in fluid communication in the bladder 28 by an air line 32 may be included to pump air into the inflatable bladder. A one-way valve (not shown) may be disposed in either the air line 32 or the bladder 28 to achieve the delivery of air into the bladder. A release valve 34 may be provided to release air from the inflatable bladder 28.

The arrangement is such that the pressure application assembly 20 is wrapped around the patient's leg so that the device 10 is disposed to engage the sciatic nerve of the patient in the manner and position in FIG. 2. Once properly positioned, the belt 22 is tightly secured so that the hook and loop fastener material 26 maintains the belt in place. If further tightening is required to increase the application of pressure on the patient's sciatic nerve, the bulb 30 may be squeezed by hand to increase the volume of air in the air bladder 26 much in the same manner as a blood pressure reading apparatus. This squeezing action results in the tightening of the belt 22 around the patient's leg, which increases the force of the device against the sciatic nerve. The patient (or the health care professional, as the case may be) may then apply the device for a desired period of time to relieve pain experienced by the patient.

FIGS. 4-7 illustrate devices 50, 60, 70 and 80 of further embodiments. As shown, the engaging surfaces are relatively flat and small in surface area. As shown, the surface area of the device may be configured in any shape and form. For example, circular-shaped surfaces (device 50), triangular-shaped surfaces (device 60), diamond-shaped surfaces (device 70) and hexagon-shaped surfaces (device 80) may be employed. Each device has a small engaging surface so that when it is applied to a patient's sciatic nerve, the device provides localized, discrete pressure on the nerve.

The foregoing indicates that the methods and devices of relieving pain in a patient of embodiments of the invention provide a rapid, convenient and powerful way to overcome pain. The methods of relieving pain disclosed herein are capable of relieving pain from different kinds of pathologies, and also relieving discomfort from other pathologies, such as cancer, belly floating, high fever and chemotherapy, to name a few. However, the pain is intended to indicate to a person when something is wrong with the body. The methods and devices disclosed herein are for the immediate relief of pain and not necessarily for curing the underlying disease or for completely eliminating pain; although with certain diseases, the complete elimination of pain may occur.

The methods and devices may be applied by individuals requiring minimal training. The methods and devices may be employed when a patient is not responding to other treatment, e.g., acupuncture, or when a patient wants to avoid using medicine.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of providing relief from pain in a patient, the method comprising applying direct pressure on at least one of the sciatic nerves of the patient.

2. The method of claim 1, further comprising applying pressure for a period of time.

3. The method of claim 2, wherein pressure is applied for a period of time extending between twenty seconds and ten minutes.

4. The method of claim 1, wherein relief from pain lasts at least one-half an hour.

5. The method of claim 1, applying direct pressure on at least one of the sciatic nerves of the patient may be achieved by using a person's hand.

6. The method of claim 1, applying direct pressure on at least one of the sciatic nerves of the patient may be achieved by utilizing a device having a small surface.

7. The method of claim 1, wherein the method is particularly suited for relieving pain caused by a medical condition, and wherein the medical condition may be selected from a group consisting of a renal condition, a dental condition and a tumor condition.

8. The method of claim 1, further comprising applying direct pressure on the other of the sciatic nerves of the patient.

9. A method of providing relief from pain in a patient caused by a medical condition, wherein the method comprises:
applying a first pressure relieving device on at least one of the sciatic nerves of the patient;
manipulating the first pressure relieving device to increase the pressure on the at least one of the sciatic nerve of the patient; and
applying the first pressure relieving device at an increased pressure for a period of time.

10. The method of claim 9, further comprising:
applying a second pressure relieving device on the other of the sciatic nerves of the patient;
manipulating the second pressure relieving device to increase the pressure on the other of the sciatic nerves of the patient; and
applying the second pressure relieving device at an increased pressure for a period of time.

11. The method of claim 10, wherein the medical condition may be selected from a group consisting of a renal condition, a dental condition and a tumor condition.

12. The method of claim 9, wherein the device is configured to apply pressure on the sciatic nerve.

13. The method of claim 12, wherein the device comprises a small surface.

14. A pressure application device configured to apply pressure to a sciatic nerve of the patient, the pressure application device comprising a small surface to engage the sciatic nerve of the patient.

15. The pressure application assembly of claim 14, wherein the small surface is configured in the shape of an arc.

16. A pressure application assembly comprising:
a belt configured to wrap around a leg of a patient; and
a device coupled to the belt, the device being configured to apply pressure to a sciatic nerve of the patient.

17. The pressure application assembly of claim 16, further comprising an apparatus, coupled to the belt, to increase the application of pressure of the device against the sciatic nerve of the patient.

18. The pressure application assembly of claim 17, wherein the apparatus comprises an inflatable bladder and a bulb in fluid communication with the inflatable bladder to deliver air under pressure to the inflatable bladder.

19. The pressure application assembly of claim 16, wherein the device comprises a small surface.

20. The pressure application assembly of claim 19, wherein the small surface is configured in the shape of an arc.
